(19) United States
(12) Patent Application Publication
(10) Pub. No.: US 2025/0271456 A1
(43) Pub. Date: Aug. 28, 2025

(54) MULTI-LAYER SOFTWARE ARCHITECTURE FOR OPERATING DIAGNOSTIC LABORATORY SYSTEMS

(71) Applicant: Siemens Healthcare Diagnostics Inc., Tarrytown, NY (US)

(72) Inventors: Klaus Kirchberg, Plainsboro, NJ (US); Rayal Prasad, Princeton, NJ (US); Ankur Kapoor, Plainsboro, NJ (US)

(73) Assignee: Siemens Healthcare Diagnostics Inc., Tarrytown, NY (US)

(21) Appl. No.: 18/857,925

(22) PCT Filed: May 4, 2023
(86) PCT No.: PCT/US2023/020984

§ 371 Date: Oct. 18, 2024

Related U.S. Application Data
(60) Provisional application No. 63/364,258, filed on May 5, 2022.

(51) Int. Cl. G01N 35/00; G01N 35/04

(52) U.S. Cl. CPC G01N 35/0092; G01N 35/00732; G01N 35/04; G01N 2035/00326; G01N 2035/0401

(57) ABSTRACT

A diagnostic laboratory system includes one or more instruments and a transport system configured to transport sample containers to and from the one or more instruments, wherein each of the sample containers contains a biological sample. A computer is configured to execute a program to control operation of the diagnostic laboratory system. The program has an architecture comprising a plurality of individually replaceable software modules, wherein one or more software modules identify at least one test or plan at least one phase thereof to be performed on the biological sample. Another software module generates instructions to cause one or more of the sample containers to move to or from the one or more instruments in accordance with the at least one test or the at least one phase thereof. Methods of operating a diagnostic laboratory system are also disclosed.

### CROSS REFERENCE TO RELATED APPLICATION

This application claims the benefit of U.S. Provisional Patent Application No. 63/364,258, entitled “MULTI-LAYER SOFTWARE ARCHITECTURE FOR OPERATING DIAGNOSTIC LABORATORY SYSTEMS” filed May 5, 2022, the disclosure of which is hereby incorporated by reference in its entirety for all purposes.

### FIELD

This disclosure relates to software architecture for operating diagnostic laboratory systems.

### BACKGROUND

Diagnostic laboratory systems conduct tests to identify analytes or other constituents in biological samples such as blood serum, blood plasma, urine, interstitial liquid, cerebrospinal liquids, and the like. The samples may be received in and/or transported throughout the laboratory systems in sample containers. The samples are typically routed to a plurality of different instruments of the laboratory system to be processed and analyzed.

Many laboratory systems route the samples to their destinations based on a heuristic rule set that decides which route each sample container should take. This routing technique can lead to inefficient travel of the sample containers that may include traffic jams of sample containers throughout the laboratory systems. Current sample container routing programs are not flexible and thus need substantial customization for each new laboratory layout or substantial updating to accommodate hardware changes to existing laboratory layouts, which can be both time consuming and expensive. Therefore, a need exists for systems and methods of operating diagnostic laboratory systems and transporting sample containers that provide flexibility for different laboratory configurations and hardware changes thereto.

### SUMMARY

According to a first aspect, a diagnostic laboratory system for analyzing a biological sample is provided. The diagnostic laboratory system includes at least one instrument for preparing or testing the biological sample, and a transport system configured to transport a sample container to and from the at least one instrument. The sample container contains therein the biological sample to be analyzed, and the transport system comprises a plurality of sample carriers, track segments, and track switches. The diagnostic laboratory system also includes a computer configured to execute a program to control operation of the diagnostic laboratory system. The program has an architecture comprising a plurality of individually replaceable software modules in communication with each other. The plurality of individually replaceable software modules includes a first software module to identify at least one test or plan at least one phase thereof to be performed on the biological sample. The plurality of individually replaceable software modules also includes a second software module to generate instructions to the transport system to cause the sample container to move to or from the at least one instrument in accordance with the at least one test or the at least one phase thereof, wherein an electrical specification change of a hardware component of the transport system requires only the second software module to be updated.

In another aspect, a method of operating a diagnostic laboratory system for analyzing a biological sample is provided. The method includes identifying at least one test to be performed on the biological sample using a first software module in response to receiving an input at the first software module. The input is received from a workstation of the diagnostic laboratory system or an external source. The first software module is part of a program comprising a plurality of individually replaceable software modules in communication with each other, and the program is executing on a computer to control operation of the diagnostic laboratory system. The method also includes planning at least one phase of the at least one test using the first or another software module of the program, and generating instructions to a transport system of the diagnostic laboratory system using a second software module of the program to cause a sample container containing the biological sample to move to or from an instrument of the diagnostic laboratory system in accordance with the at least one test or the at least one phase thereof, wherein an electrical specification change of a hardware component of the transport system requires only the second software module to be updated. The method further includes transporting the sample container using the transport system in response to the generated instructions.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. This disclosure is intended to cover all modifications, equivalents, and alternatives falling within the scope of the claims and their equivalents.

### BRIEF DESCRIPTION OF THE DRAWINGS

The drawings, described below, are provided for illustrative purposes, and are not necessarily drawn to scale. Accordingly, the drawings and descriptions are to be regarded as illustrative in nature, and not as restrictive. The drawings are not intended to limit the scope of the disclosure in any way.

FIG. 1A illustrates a block diagram of a diagnostic laboratory system including a plurality of instruments and a plurality of track segments connecting the instruments according to one or more embodiments.

FIG. 1B illustrates an example transport system for the laboratory system of FIG. 1A in accordance to one or more embodiments.

FIG. 2 illustrates a route map showing routes that sample containers may take within a laboratory system according to one or more embodiments.

FIG. 3 illustrates a laboratory system that has a different configuration and more instruments than the laboratory system of FIG. 1A according to one or more embodiments.

FIG. 4 illustrates software layers and their relations to each other, wherein the software layers can operate independently of each other according to one or more embodiments.

FIG. 5 illustrates individually replaceable software modules that may implement the layers of FIG. 4 according to one or more embodiments.

FIG. 6 illustrates a flowchart of a method of operating a diagnostic laboratory system according to one or more embodiments.

### DETAILED DESCRIPTION

Diagnostic laboratory systems conduct clinical chemistry and/or assays to identify analytes or other constituents in biological samples such as blood serum, blood plasma, urine, interstitial liquid, cerebrospinal liquids, and the like. The biological samples are collected in sample containers and transported via the sample containers to instruments throughout the laboratory system where the samples are processed and analyzed. For example, the instruments may prepare the samples for tests and then conduct the tests on the samples.

Conventional laboratory systems route the samples to their destinations based on heuristic rule sets that decide which route each respective sample container should take. These heuristic rule sets can cause unnecessary travel for the sample containers, which makes the laboratory systems inefficient.

The embodiments disclosed herein overcome problems with sample processing and transport issues of conventional laboratory systems. The laboratory systems described herein use dynamic routing algorithms implemented in software having an architecture that is divided into multiple software layers. The layers may be designed, developed, tested, and executed independently of each other, which enables the same architecture to be easily scaled for a wide variety of laboratory configurations.

In some embodiments, the multi-layer software architecture abstracts a logical set of planning tasks and/or workflow to be accomplished from a physical transport layer that activates hardware components. By separating the layers, intelligent routing may be performed to increase the efficiency of the laboratory system without affecting other tasks and/or workflow performed by other layers. In addition, the separation of the layers may include clear interfaces, which enable replacing or revising a first layer without affecting the other layers. Layers are software components of the architecture that are sequentially interconnected, can exchange inputs and outputs with an immediate downstream and/or upstream layer, and can execute independently in parallel. For example, a first layer may execute with a first set of inputs independently of a second layer that may execute concurrently with a second set of inputs received from the first layer.

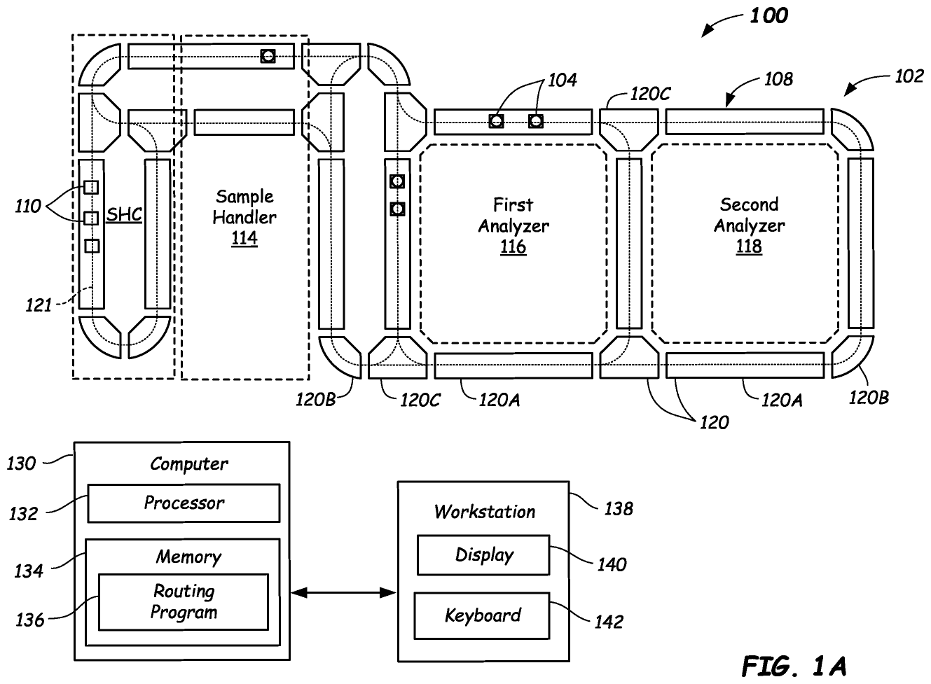

These and other embodiments of laboratory systems and method of operating laboratory systems are described in greater detail with reference to FIGS. 1A-6.

Reference is made to FIG. 1A, which illustrates a block diagram of an embodiment of a diagnostic laboratory system 100. The laboratory system 100 may include a plurality of instruments 102 configured to process biological samples and sample containers 104 (a few labelled) and to conduct assays or tests on the samples. The samples may be any biological specimen collected from, e.g., a person or animal. The samples may be collected in the sample containers 104 and delivered to the laboratory system 100, wherein they may be transported throughout the laboratory system 100, such as to different ones of the instruments 102. In the embodiment of FIG. 1A, the system 100 has three instruments 102, which include a sample handler 114, a first analyzer 116, and a second analyzer 118. The laboratory system 100 may include more instruments than shown in FIG. 1A.

The sample containers 104 with the samples contained therein may be transported between one or more of the instruments 102 in the laboratory system 100. The laboratory system 100 may include a track 108 configured to move sample carriers (carriers 110—a few labelled) to and from the instruments 102. The sample containers 104 may be received in the carriers 110 so as to be moved throughout the laboratory system 100 on the track 108.

In some embodiments, the track 108 may extend proximate or around at least some of the instruments 102 as shown in FIG. 1A. As described herein, portions of the instruments 102 may have devices, such as robots (not shown in FIG. 1A), that transfer sample containers 104 to and from the carriers 110 located on the track 108. For example, the sample handler 114 may move the sample containers 104 to empty carriers 110 that are moved to specific locations on the track 108 after the sample containers 104 are received in the laboratory system 100. The sample handler 114 may also remove the sample containers 104 from the carriers 110 when the carriers return to specific locations on the track 108. One or more of the instruments 102 may remove samples from the sample containers 104 when the sample containers 104 are located at the specific locations on the track 108.

As shown in FIG. 1A, the track 108 may include a plurality of segments 120 (a few labelled) that may be interconnected. The segments 120 may include straight segments 120A, curved segments 120B, and switch segments 120C. The switch segments 120C may merge different ones of the segments 120 together or the switch segments 120C may include switches (not shown) that enable the sample containers to be routed to at least a first track segment or a second track segment. The carriers 110 may move in the segments 120 as shown by the small dashed lines 121. In some embodiments, some of the segments 120 may be integral with one or more of the instruments 102. The instruments 102 may be arranged in a plurality of configurations and have access to the track 108.

Diagnostic laboratory systems, such as the laboratory system 100, may have many instruments and may have tracks linked to other laboratory systems. The laboratory systems, including the laboratory system 100, may simultaneously move and process a plurality of carriers 110 and their respective sample containers. In some embodiments, the laboratory system 100 or other laboratory systems may move and process hundreds or thousands or carriers 110 and their respective sample containers simultaneously.

The track 108 may be part of a transport system configured to transport sample containers throughout the laboratory system 100. The transport system may also include the sample handler 114. In some embodiments, the transport system may include other components configured to transport the sample containers. These components may include motors on the track 108 or the carriers 110 that can move the carriers 110 relative to the track 108. In some embodiments, the components may also include switches and the like that guide the sample containers 104 on one or more paths on the track 108. These components may be activated by power sources/supplies and the like and may be controlled by a computer (e.g., computer 130) as described herein.

FIG. 1B illustrates an example transport system 122 for laboratory system 100 of FIG. 1A in accordance with embodiments provided herein. As shown in FIG. 1B, in some embodiments, transport system 122 may include sample handler 114 as well as other components configured to transport the sample containers. These components may include motors on the track 108 or the carriers 110, such as motor 124, that can move the carriers 110 relative to the track 108. In some embodiments, the components may also include switches (such as switch 126) that guide the sample containers 104 on one or more paths on the track 108. Other example components include sensors for detecting carrier 110 (e.g., sensor 128). The various components of transport system 122 may be activated by power sources/supplies (such as power source 130) and the like and may be controlled by a computer (e.g., computer 130) as described herein.

The laboratory system 100 may include or be coupled to computer 130 configured to execute a program that is configured to control operation of the laboratory system 100. The computer 130 may be configured to communicate with the instruments 102 and other components of the laboratory system 100, such as components in the transport system. The computer 130 may include a processor 132 that is configured to execute programs, such as computer code, including the software described herein.

The computer 130 may include or have access to memory 134 that may store one or more programs (e.g., software) and/or data. The memory 134 may include a routing program 136 configured to operate the laboratory system 100 and/or route the carriers 110 and, thus the sample containers 104, throughout the laboratory system 100. The routing program 136 may include a plurality of different programs as described herein. Other programs may analyze data generated by the instruments 102. The computer 130 may be coupled to a workstation 138 that enables users to interface with the laboratory system 100. The workstation 138 may include a display 140 and a keyboard 142. In some embodiments, the memory 134, the routing program 136, and/or other programs may be referred to as a non-transitory computer-readable medium comprising computer code executable on the processor 132. In some embodiments, local and/or remote computers, workstations, and/or memories may be employed.

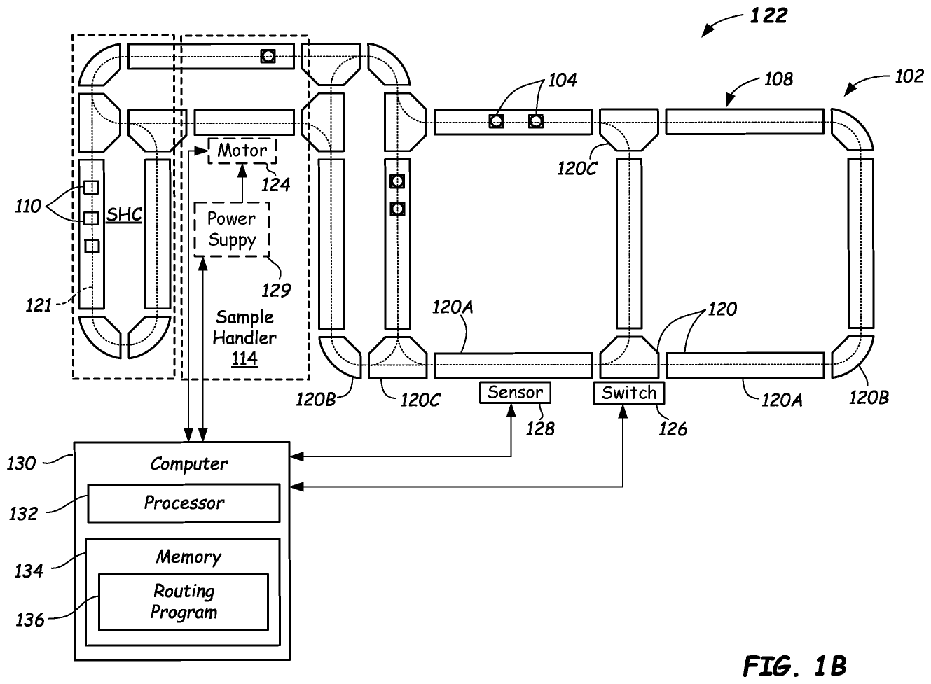

Additional reference is made to FIG. 2, which illustrates a route map 220 showing routes or paths that the sample containers 104 may take within the laboratory system 100. The route map 220 corresponds with the small dashed lines 121 in FIG. 1A. As shown in FIG. 2, the carriers 110 (a few labelled in FIG. 2) may take many routes throughout the laboratory system 100. The routing program 136 that operates the laboratory system 100 routes the carriers 110 to the proper instruments at scheduled times to keep the laboratory system 100 operating efficiently. In some embodiments, the routing program 136 may determine the most efficient path for one or more of the carriers 110 by taking into account that there may be other carriers travelling on the same path.

The laboratory system 100 has various locations on the track 108 where actions may be performed. In some embodiments, the locations may have components controlled by the routing program 136. One or more of the locations may include a pick and place station 222 that may be configured to transfer the sample containers 104 between the sample handler 114 and the carriers 110. The locations may also include one or more cameras 224 configured to capture images of the sample containers 104 and/or the carriers 110 such as at the pick and place station 222.

Some of the locations may be proximate or within the first analyzer 116 and may include a first aspiration station 216. Other locations may be proximate or within the second analyzer 118 and may include a second aspiration station 218. The first aspiration station 216 and the second aspiration station 218 may be locations where the sample containers 104 may be transported to so that the first aspiration station 216 and the second aspiration station 218 may aspirate samples from the sample containers 104. The first analyzer 116 and the second analyzer 118 may then process the samples per test orders that may be received in the laboratory system 100. The sample containers 104 may be routed such that they arrive at the first aspiration station 216 and the second aspiration station 218 at specific times as determined by the routing program 136 to ensure efficient operation of the laboratory system 100.

The track 108 may include one or more locations implemented as input queues (IQ) where the sample containers 104 may be queued prior to entering a station or an instrument. The sample containers 104 may move in both directions through the first aspiration station 216 and the second aspiration station 218, so the first analyzer 116 and the second analyzer 118 may have input queues IQ on both sides of the first aspiration station 216 and the second aspiration station 218. Each of the input queues IQ may be associated with input process queues IPQ and/or exit queues EQ. The track 108 may have one or more locations implemented as working queues WQ that may queue the sample containers 104 as they wait to be processed. The sample handler 114 may be associated with one or more locations implemented as on-deck queues (ODQ) that may queue the carriers 110 to receive sample containers 104.

The routing program 136 may generate instructions that cause the carriers 110 to move on the track 108 throughout the laboratory system 100. For example, components in the transport system may activate in response to the instructions, which may cause the sample containers 104 to move on the track 108. For example, electric current may be applied to motors in the carriers 110 and/or switches in the switch segments 120C. In some embodiments, the routing program 136 may receive instructions indicating the tests that are to be performed on each of the samples and whether there is a priority for the testing. For example, the instructions may be received from a hospital information system (not shown) or another external source. Instructions may also be received from a system operator using workstation 138.

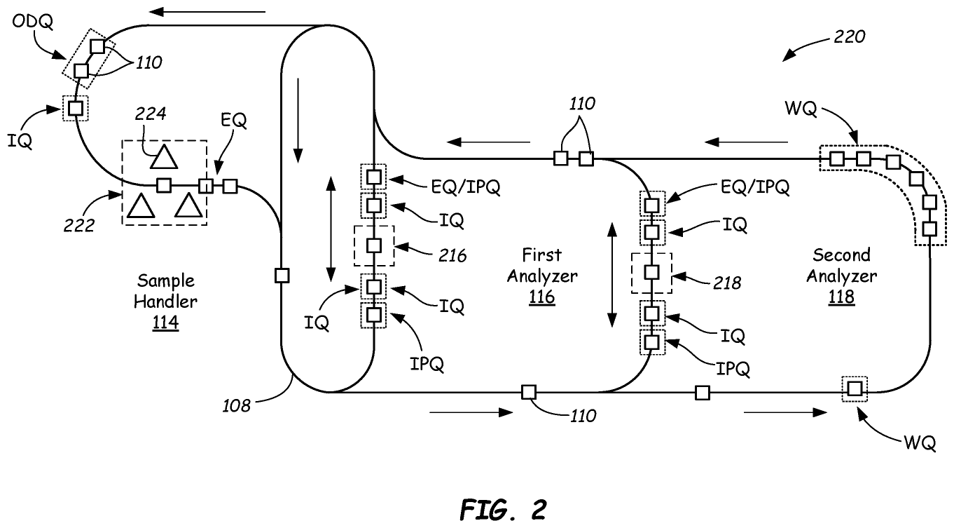

Additional reference is made to FIG. 3, which illustrates a laboratory system 300 that has a different configuration than the laboratory system 100 of FIG. 1A. The laboratory system 300 includes more instruments 302 than the laboratory system 100, and the instruments 302 are arranged differently than the instruments 102 in FIG. 1A. The laboratory system 300 includes a sample handler 314, a first analyzer 316, a second analyzer 318, a third analyzer 320, and a fourth analyzer 322 arranged in an L-shape. Thus, the routing of the carriers 110 in the laboratory system 300 may be much more complex than the routing of the carriers 110 in the laboratory system 100. In conventional laboratory systems, the routing program may be different for every configuration of a laboratory system. The routing program 136 described herein may include a plurality of software layers implemented in a plurality of individually replaceable software modules that enable the routing program 136 to be easily modified to operate other laboratory systems including the laboratory system 300. Thus, unlike the routing programs used in conventional laboratory systems, the routing program 136 described herein may be used for many different laboratory configurations.

The components in laboratory systems, including the laboratory system 100 and the laboratory system 300, can be broadly characterized as including a transport system, the carriers 110, and the instruments 102/302. The transport system includes the track 108 and other hardware that move the carriers 110. The carriers 110 transport the sample containers 104 throughout the laboratory systems 100/300. The instruments 102/302 may include analyzers and modules to which the carriers 110 are directed by the routing program 136.

Returning to FIG. 1A, when a sample container 104 is introduced to the laboratory system 100, the sample container is loaded into one of the carriers 110. The carriers 110 are instructed to go to specific sets of destinations depending on the processes and/or tests that are to be performed on the samples in the sample containers 104. The destinations may include the instruments 102, which may include, e.g., centrifuges, chemistry analyzers, decappers, storage/refrigeration units and other components. The routing between the destinations may be in a particular sequence. For example, a sample container may need to travel to a centrifuge first followed by a decapper. In addition, a sample container may need to be transported to one of the instruments 102 within a specific time window. For example, the sample container may be instructed to be aspirated within a certain time window.

One of the technical challenges of operating laboratory systems is achieving a high throughput of samples with minimal human interaction while ensuring accuracy of all the tests regardless of the configuration of the laboratory system. The routing program 136 described herein accommodates different sizes and configurations of laboratory systems without requiring a complete re-engineering of the entire routing program 136. In addition to the foregoing, fault tolerance in carrier transport is also an important factor to ensure a reliable workflow. The routing program 136 described herein has a plurality of software layer functions that may be constant between different laboratory systems. The layers have been designed or programmed to operate on all or most laboratory systems.

In some embodiments, the routing program 136 is a multi-layer software architecture that abstracts a logical set of tasks and/or workflow to be accomplished by a physical transport software layer. Each software layer executes independently of other software layers but may transmit data to and/or receive data from other independently executing software layers. Thus, a software layer may be changed or customized without necessarily affecting other software layers. By separating the layers, intelligent routing can be performed to increase the efficiency of the laboratory systems 100, 300. In addition, the separation of layers with clear interfaces allows replacing a layer without affecting the other layers. In some embodiments, one or more software layers may each plan/determine at least one phase (e.g., a pre-or post-process action) of a test to be performed by at least one of the instruments 102, another software layer may determine at least one routing of a sample container 104 to or from the one or more instruments 102 to perform that at least one phase, and still another software layer may generate instructions to hardware components (e.g., motors, power supplies, switches, sensors, etc.) to carry out the at least one routing of the sample container 104.

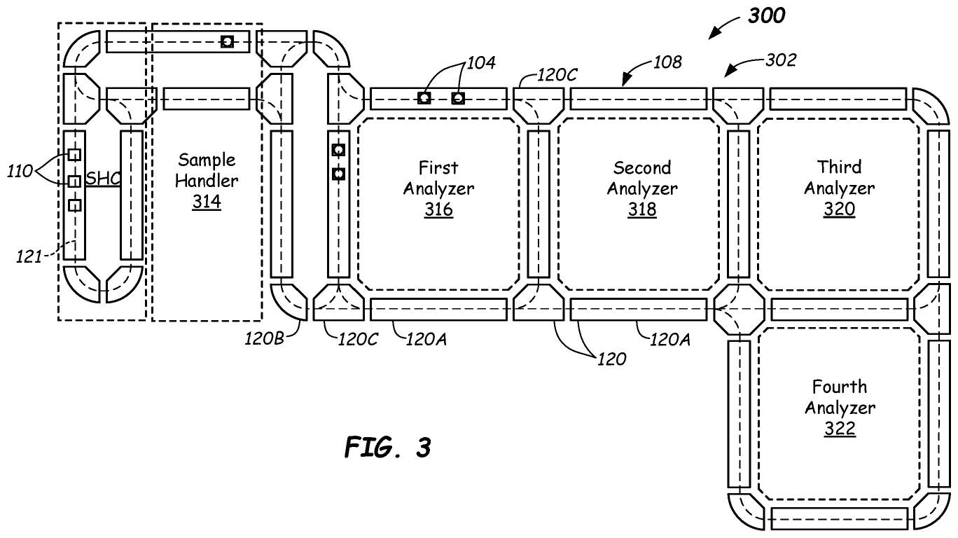

Reference is made to FIG. 4, which illustrates an example of software layers 400 that may be used in the architecture of the routing program 136 in accordance with embodiments provided herein. The architecture may include other layers, more layers, or fewer layers than are illustrated in FIG. 4. An order layer 402 may receive sample analysis orders and in response identify specific tests available in laboratory system 100 to be performed on the associated biological samples to fulfill the sample analysis orders. In some embodiments, order layer 402 may receive sample analysis orders from sources external to the laboratory system 100, such as a hospital information system (not shown). Order layer 402 may additionally receive sample analysis orders entered by a system operator of laboratory system 100 via workstation 138. Based on the received inputs, the order layer 402 may generate an output (e.g., instructions) that indicate what tests are to be performed. For example, a first sample may require tests A, B, and C; a second sample may require tests X, Y, and Z; and a third sample may require only test A. Each of tests A, B, C, X, Y, and Z can be performed by one or more of the instruments 102 of the laboratory system 100. For example, the first analyzer 116 may be configured to perform tests A, B, and Z and the second analyzer 118 may be configured to perform tests C, X, and Y.

A task layer 404 may be configured to receive the output data generated by the order layer 402 and may generate an output (e.g., instructions) regarding the individual tasks required to perform the tests identified by the order layer 402. For example, the task layer 404 may generate instructions indicating that the first sample needs to be aspirated and dispensed into another container and then mixed with a first reagent, while the second sample needs to be aspirated and dispensed into another container and then mixed with a second reagent, and the third sample needs to be aspirated and dispensed into another container and then mixed with a diluent. The task instructions are output from the task layer 404 and transmitted to an assignment layer 406 as input. In some embodiments, the task layer 404 may also transmit data back to the order layer 402 indicating that the task instructions have been generated. In some embodiments, the order layer 402 may delay its output until receiving data indicating the task layer 404 is available to process additional inputs.

The assignment layer 406 may generate an output that includes specific instructions regarding the performance of the tasks in response to receiving the output generated by the task layer 404. For example, the assignment layer 406 may generate an output that assigns a first carrier to receive the sample container containing the first sample at a specific station, such as the sample handler 114. The assignment layer 406 may also keep track of the available number of carriers for transporting sample containers and ensure that empty carriers are available where they are needed (e.g., at sample handler 114) by assigning those empty carriers to specific locations. The assignment layer 406 may further generate an output that assigns a specific location to receive the first carrier with the first sample container, such as a first aspiration station 216 of the first analyzer 116. The assignment layer 406 may additionally generate an output that assigns a second carrier to receive the sample container containing the second sample at a specific location and that assigns a second aspiration station 218 of the second analyzer 118 to receive the second carrier. The assignment layer 406 may further generate an output that assigns, e.g., the first aspiration station 216 of the first analyzer 116 to receive a third carrier carrying a sample container containing the third sample, and the like. In the event that a test on a sample is urgent, the assignment layer 406 may generate an output assigning the carrier carrying the urgent sample to pass other less urgent samples. The assignment instructions are output from the assignment layer 406 and transmitted to a trajectory layer 408 as input. In some embodiments, the output generated by the assignment layer 406, or data indicating that the assignment layer 406 has generated an output, may also be transmitted back to the task layer 404 or other layers 400. In some embodiments, the task layer 404 may delay its output until receiving data indicating that the assignment layer 406 is available to process additional inputs.

The trajectory layer 408 generates an output that includes specific paths for the carriers 110 to follow in response to receiving the output generated by the assignment layer 406. For example, referring to FIG. 2, the trajectory layer 408 may generate an output indicating that specific ones of the carriers 110 are to be transported via specific paths (e.g., segments 120 of the track 108) to specific locations. The output of trajectory layer 408 may include respective carrier paths that prevent the carriers 110 from colliding with each other. The trajectory layer 408 may also optimize paths of the carriers 110 such that the carriers 110 move to and from locations on the track 108 as efficiently as possible. In some embodiments, the trajectory layer 408 may indicate that high priority samples be moved before other samples as described above. In addition, the trajectory layer 408 may determine which segments 120 of the track 108 each of the carriers will travel on. Also, the trajectory layer 408 may determine a schedule or time window at or within which each of the carriers will travel on specific ones of the segments 120. These determinations may avoid traffic jams and/or collisions in the transport system. The trajectory output is transmitted to a physical transport layer 410 as input. In some embodiments, the trajectory output or data relating thereto may be transmitted back to the assignment layer 406 or other layers 400. For example, in some embodiments, the assignment layer 406 may delay its output until receiving data indicating that the trajectory layer 408 is available to process additional inputs. Also, receiving at the assignment layer 406 the trajectory output or data relating thereto of a first carrier may affect a subsequent assignment of a second carrier. For example, the assignment layer 406 may avoid assigning the second carrier to certain locations or instruments 102 to avoid congestion or collisions knowing that the first carrier has a carrier path taking the first carrier past those certain locations or instruments 102. The assignment layer 406 may thus generate more efficient assignment instructions for the second carrier in response to receiving trajectory instructions for the first carrier.

The physical transport layer 410 may generate instructions to the transport system (e.g., transport system 122 of FIG. 1B) that may activate hardware components in the transport system to cause selected carriers 110 to move on the track 108 in response to the paths determined by the trajectory layer 408. The hardware components may include sample carriers, track segments, track switches, motors, power supplies, power sources, sensors, transformers, and/or other electrical circuit parts. Other hardware components of the transport system may be activated to move the carriers 110 pursuant to the carrier paths determined by the trajectory layer 408. For example, the physical transport layer 410 may generate instructions to the transport system that cause selected switches in the switch segments 120C to be appropriately set to form the determined carrier path and to cause the selected carriers 110 to move to or from a specific location (e.g., an instrument 102). That is, the instructions may cause appropriate electrical power/current/voltage to be applied to e.g., selected motors in the identified carriers 110 (or track segments 120 in those embodiments with movable track segments) and/or the identified switches in the switch segments 120C to cause the carriers 110 to move along the specific paths on the track 108 determined by the trajectory layer 408. In some embodiments, the physical transport layer 410 may transmit data back to the trajectory layer 408 and/or other layers 400 indicating that the physical transport layer 410 has generated the instructions.

In sum, each of the software layers 402, 404, 406, and 408 may be considered a “planning” layer that provides input to its next downstream software layer 400 (e.g., the order layer 402 provides input to the task layer 404, which provides input to the assignment layer 406, which provides input to the trajectory layer 408), while the physical transport layer 410 may be considered an “executing” layer that puts the sample containers in motion in the laboratory system 100. Routing program 136 and software layers 400 also may be employed with laboratory system 300 of FIG. 3.

In some embodiments, each of the layers 404, 406, 408, and 410 may provide feedback up to at least its nearest upstream layer 400 to (a) alert the upstream layer of its availability and (b) enable that upstream layer, where possible, to more efficiently process inputs received from its nearest upstream layer (e.g., the physical transport layer 410 may provide input to the trajectory layer 408 to enable the trajectory layer 408 to more efficiently process an input received from the assignment layer 406). That is, by receiving input from a downstream layer, a layer 400 may generate alternative plans and/or instructions affecting the tests to be performed by the laboratory system 100 (or laboratory system 300) than it would without the input from the downstream layer. The generated alternative plans and/or instructions may, e.g., avoid delays in placing sample containers 104 in carriers 110, avoid traffic congestion at specific track segments 120 and/or instruments 102, and/or avoid carrier collisions that may improve overall sample testing throughput.

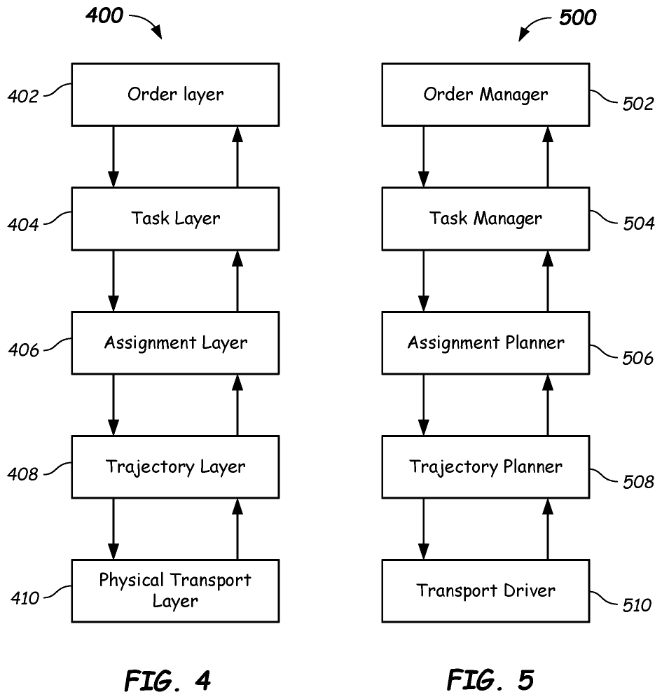

Additional reference is made to FIG. 5, which illustrates an embodiment of the routing program 136 partitioned as separate and individually replaceable software modules 500, each of which respectively implements one of the layers 400 in accordance with one or more embodiments. Software modules 500 may include an order manager 502 that implements the order layer 402, a task manager 504 that implements the task layer 404, an assignment planner 506 that implements the assignment layer 406, a trajectory planner 508 that implements the trajectory layer 408, and a transport driver 510 that implements the physical transport layer 410. In some embodiments, communications between the software modules 500 (and thus the layers 400) may be performed, e.g., via a message bus or a data-centric distributed data service (DDS). Other communications protocols may be used. Each of the software modules 500 may reside in and be executed in a centralized fashion by one controller, such as, e.g., computer 130 (FIG. 1A), or in a distributed fashion, wherein one or more of the software modules 500 may reside in and be executed in one or more respective sub-controllers (each having its own memory) or may be executed in one or more respective sub-processors of processor 132 of computer 130. Segmentation of the routing program 136 processing may also be performed geographically, such as on certain parts of the track 108. Segmentation of the processing may also assign each sub-controller to a certain group of samples and/or a certain group of the carriers 110. Segmentation of the processing may be performed on other groups of samples based on, e.g., the particular tests to be performed. The routing program 136 may include software modules other than the modules 500 shown in FIG. 5.

The modules 500 may execute the layers 400 independent of and/or in parallel with each other. For example, the transport driver 510 may send electrical signals to motors, drivers, switches, and other hardware components of the transport system to perform required motions of one or more carriers 110 pertaining to one or more first tests or phases thereof. Independently of and/or in parallel with transport driver 510, the trajectory planner 508 may plan paths for one or more carriers 110 pertaining to one or more second tests or phases thereof so that these carriers 110 may reach their destinations in minimal time while avoiding collisions and traffic congestion. Independently of and/or in parallel with transport driver 510 and the trajectory planner 508, the assignment planner 506 may assign available carriers 110 to sample containers pertaining to one or more third tests or phases thereof. Independently of and/or in parallel with the transport driver 510, the trajectory planner 508, and the assignment planner 506, the task manager 504 may break down one or more fourth tests into discrete tasks or phases to be performed in order to complete the one or more fourth tests. And independently of and/or in parallel with transport driver 510, the trajectory planner 508, the assignment planner 506, and the task manager 504, the order manager 502 may process a sample analysis order into one or more fifth tests received from a system operator or one or more users, such as doctors and other medical professionals in communication with the laboratory system 100 (or laboratory system 300).

As described above in connection with layers 400, each of the software modules 502, 504, 506, and 508 may receive, in addition to inputs received from an upstream module 500, optional inputs from one or more downstream modules 504, 506, 508, and 510 that may be used in the execution of their respective layer 400. In some embodiments, the inputs may include status of a module 500 or data generated by a module 500. For example, status or data generated by the transport driver 510 may be transmitted back to the trajectory planner 508 and/or other modules 500. Status or data generated by the trajectory planner 508 may be transmitted back to the assignment planner 506 and/or other modules 500. Status or data generated by the assignment planner 506 may be transmitted back to the task manager 504 and/or other modules 500. And status or data generated by the task manager 504 may be transmitted back to the order manager 502.

Separating the routing program 136 into multiple modules/layers advantageously makes the system more scalable, allowing small, medium, and large laboratory systems to run on the same software platform. In some embodiments, the routing program 136, divided into multiple modules 500/layers 400, enables the routing program 136 to be used on a plurality of different laboratory system configurations, such as the laboratory system 100 and the laboratory system 300. In addition, the multiple layers in the routing program 136 may be designed, developed, and tested independently. Thus, if one of the modules 500/layers 400 needs to be edited or replaced, the revisions may not affect the other modules 500/layers 400. For example, if laboratory system 100 expands to include an additional instrument 102 that performs a same function as existing instruments 102 (to increase sample analysis throughput), order manager 502 and task manager 504 may not need to be updated. If laboratory system 100 expands to include additional track segments 120 and/or track switches (e.g., to alleviate traffic congestion), order manager 502, task manager 504, and assignment planner 506 may not need to be updated. And if an electrical specification change of a hardware component of the transport system in laboratory system 100 occurs (e.g., due to a replacement or upgrade of a hardware component), only the transport driver 510 may need to be updated, because only the physical transport layer 410 generates instructions to activate hardware components in the transport system to cause selected sample carriers (and any sample containers carried therein) to move. The electrical specification change may include. for example, changes to any hardware component's electrical power, current, and/or voltage requirements (e.g., for motor 124, switch 126, sensor 128, power supply 129 of FIG. 1B or the like); carrier and/or track motor details affecting carrier acceleration and speed on track 108; timing issues related to powering up and down of hardware components; track sensor issues affecting allowable distances between moving carriers, etc.

Note that a software module 500 may include more than one software layer 400. For example, in some embodiments, software modules 500 may include only software modules 502 and 510, wherein software module 502 may include software layers 402, 404, 406, and 408 (i.e., the “planning” layers) while software module 510 may include software layer 410 (i.e., the “executing” layer). In other embodiments, software modules 500 may include only software modules 502, 508, and 510, wherein software module 502 may include software layers 402, 404, and 406. Software module 508 may include trajectory layer 408, and software module 510 may include physical transport layer 410. In still other embodiments, software modules 500 may include only software modules 502, 506, 508, and 510, wherein software module 502 may include software layers 402 and 404. Other embodiments may include other software modules 500 that, e.g., may analyze test results or perform various pre- and/or post processing functions.

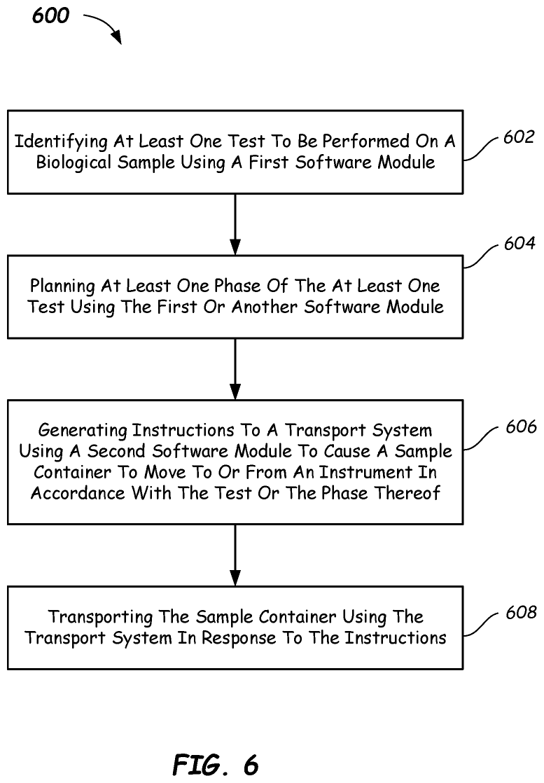

Reference is now made to FIG. 6, which illustrates a flowchart showing a method 600 of operating a diagnostic laboratory system (e.g., laboratory system 100). The method 600 includes, at process block 602, identifying at least one test to be performed on a biological sample using a first software module. For example, the first software module may be order manager 502 executing order layer 402. In response to receiving a sample analysis order, order manager 502 may identify which laboratory system 100 tests are to be performed on the associated biological sample.

The method 600 also includes, at process block 604, planning at least one phase of the test using the first or another software module. For example, the at least one phase may be planned by a software module 500, such as by task manager 504 that includes the task layer 404, and may include an aspiration of the biological sample from the sample container. In another example, the at least one phase may be planned by a software module 500, such as assignment planner 506 that includes the assignment layer 406, and may include an assignment of a sample carrier to receive the sample container at a specified location in the diagnostic laboratory system. And in a further example, the at least one phase may be planned by a software module 500, such as trajectory planner 508 that includes the trajectory layer 408, and may include a determination of a path over which the sample container is to be transported to or from the at least one instrument, the path including at least one track segment or at least one track switch. Other phases and/or software modules may be employed.

The method 600 further includes, at process block 606, generating instructions to a transport system using a second software module to cause a sample container to move to or from an instrument in accordance with the test or the phase thereof. For example, the second software module may be transport driver 510 that includes physical transport layer 410, which generates instructions to activate hardware components in the transport system of laboratory system 100 to move the sample container to or from the at least one instrument 102 via track 108 in accordance with the at least one test or the at least one phase thereof.

And the method 600 includes, at process block 608, transporting the sample container using the transport system in response to the instructions generated at process block 606. For example, as shown in FIG. 1 or 3, a carrier 110 carrying a sample container 104 may be transported via track 108 (e.g., via selected straight segments 120A, curved segments 120B, and switch segments 120C) to or from an instrument 102 or 302. The instructions generated at process block 606 may cause, e.g., electric currents to be applied to motors in the carrier 110 and/or switches in the switch segments 120C, which in turn may cause the carrier 110 to move on specific paths on the track 108.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure.

What is claimed is:

1. A diagnostic laboratory system for analyzing a biological sample, comprising:
at least one instrument for preparing or testing the biological sample;
a transport system configured to transport a sample container to and from the at least one instrument, wherein the sample container contains therein the biological sample to be analyzed, and the transport system comprises a plurality of sample carriers, track segments, and track switches; and
a computer configured to execute a program to control operation of the diagnostic laboratory system, the program having an architecture comprising a plurality of individually replaceable software modules in communication with each other, the plurality of individually replaceable software modules including:
a first software module to identify at least one test or plan at least one phase thereof to be performed on the biological sample, and
a second software module to generate instructions to the transport system to cause the sample container to move to or from the at least one instrument in accordance with the at least one test or the at least one phase thereof, wherein an electrical specification change of a hardware component of the transport system requires only the second software module to be updated.

2. The diagnostic laboratory system of claim 1, wherein the hardware component of the transport system includes a sample carrier, a track segment, a track switch, a motor, a power supply, a power source, a sensor, a transformer, or an electrical circuit part.

3. The diagnostic laboratory system of claim 1, wherein the first software module includes an order layer configured to identify the at least one test to be performed on the biological sample in response to input received from a workstation of the diagnostic laboratory system or an external source.

4. The diagnostic laboratory system of claim 1, wherein the first software module includes a task layer configured to plan the at least one phase thereof, wherein the at least one phase planned by task layer includes an aspiration of the biological sample from the sample container.

5. The diagnostic laboratory system of claim 1, wherein the first software module includes an assignment layer configured to plan the at least one phase thereof, wherein the at least one phase planned by the assignment layer includes an assignment of a sample carrier to receive the sample container at a specified location in the diagnostic laboratory system.

6. The diagnostic laboratory system of claim 1, wherein the first software module includes an assignment layer configured to plan the at least one phase thereof, wherein the at least one phase planned by the assignment layer includes an assignment of the at least one instrument to receive the sample container.

7. The diagnostic laboratory system of claim 1, wherein the first software module includes a trajectory layer configured to plan the at least one phase thereof, wherein the at least one phase planned by the trajectory layer includes a determination of a path over which the sample container is to be transported to or from the at least one instrument, the path including at least one track segment or at least one track switch.

8. The diagnostic laboratory system of claim 1, wherein the second software module includes a physical transport layer that generates instructions to activate hardware components in the transport system to move the sample container to or from the at least one instrument in accordance with the at least one test or the at least one phase thereof.

9. The diagnostic laboratory system of claim 1, wherein the second software module includes a physical transport layer that generates instructions to the transport system to apply appropriate electrical power to a selected one or more of the sample carriers, the track segments, or the track switches to move the sample container to or from the at least one instrument in accordance with the at least one test or the at least one phase thereof.

10. The diagnostic laboratory system of claim 1, wherein the first software module includes:
an order layer configured to identify the at least one test to be performed on the biological sample;
a task layer configured to plan a first phase of the at least one test, the first phase planned by the task layer including an aspiration of the biological sample from the sample container;
an assignment layer configured to plan a second phase of the at least one test, the second phase planned by the assignment layer including an assignment of a sample carrier to receive the sample container at a specified location in the diagnostic laboratory system; and
a trajectory layer configured to plan a third phase of the at least one test, the third phase planned by the trajectory layer including a determination of a path over which the sample container is to be transported to or from the at least one instrument, the path including at least one track segment or at least one track switch.

11. The diagnostic laboratory system of claim 1, further comprising a third software module to plan at least one phase of the at least one test and output the planned at least one phase to the second software module, the at least one phase including a determination of a path over which the sample container is to be transported to the at least one instrument, the path including at least one track segment or at least one track switch.

12. The diagnostic laboratory system of claim 11, further comprising a fourth software module to plan at least one phase of the at least one test and output the planned at least one phase to the third software module, the at least one phase including an assignment of a sample carrier to receive the sample container at a specified location and an assignment of the at least one instrument to receive the sample container.

13. The diagnostic laboratory system of claim 12, further comprising a fifth software module to plan at least one phase of the at least one test and output the planned at least one phase to the fourth software module, the at least one phase including an aspiration of the biological sample from the sample container.

14. A method of operating a diagnostic laboratory system for analyzing a biological sample, comprising:
identifying at least one test to be performed on the biological sample using a first software module in response to receiving an input at the first software module, the input received from a workstation of the diagnostic laboratory system or an external source, the first software module part of a program comprising a plurality of individually replaceable software modules in communication with each other, the program executing on a computer to control operation of the diagnostic laboratory system;
planning at least one phase of the at least one test using the first or another software module of the program;
generating instructions to a transport system of the diagnostic laboratory system using a second software module of the program to cause a sample container containing the biological sample to move to or from an instrument of the diagnostic laboratory system in accordance with the at least one test or the at least one phase thereof, wherein an electrical specification change of a hardware component of the transport system requires only the second software module to be updated; and
transporting the sample container using the transport system in response to the instructions.

15. The method of claim 14, wherein the hardware component of the transport system includes a sample carrier, a track segment, a track switch, a motor, a power supply, a power source, a sensor, a transformer, or an electrical circuit part.

16. The method of claim 14, wherein the planning comprises planning the at least one phase of the test using the first or the other software module to include an aspiration of the biological sample from the sample container.

17. The method of claim 14, wherein the planning comprises planning the at least one phase of the test using the first or the other software module to include an assignment of a sample carrier of the transport system to receive the sample container at a specified location in the diagnostic laboratory system or an assignment of the instrument of the diagnostic laboratory system to receive the sample container.

18. The method of claim 14, wherein the planning comprises planning the at least one phase of the test using the first or the other software module to include a determination of a path over which the sample container is to be transported to or from the instrument, the path including at least one track segment or at least one track switch of the transport system.

19. The method of claim 14, wherein the generating instructions comprises generating instructions using the second software module to activate hardware components in the transport system to move the sample container to or from the instrument in accordance with the at least one test or the at least one phase thereof.

20. The method of claim 14, wherein the generating instructions comprises generating instructions using the second software module to apply appropriate electrical power to a selected one or more sample carriers, track segments, or track switches of the transport system to move the sample container to or from the instrument in accordance with the at least one test or the at least one phase thereof.
